(19) United States
(12) Patent Application Publication
(10) Pub. No.: US 2025/0336484 A1
(43) Pub. Date: Oct. 30, 2025

(54) Online-coupled atmospheric chemistry transport model with data assimilation system

(71) Applicant: Institute of Atmospheric Physics, Chinese Academy of Sciences, Beijing (CN)

(72) Inventors: Ting YANG, Beijing (CN); Zifa WANG, Beijing (CN); Hongyi LI, Beijing (CN); Yifan SONG, Beijing (CN); Yining TAN, Beijing (CN); Yutong TIAN, Beijing (CN)

(21) Appl. No.: 18/894,199

(22) Filed: Sep. 24, 2024

(30) Foreign Application Priority Data
Apr. 30, 2024 (CN) 2024105436909

(51) Int. Cl. G16C 20/30; G01W 1/10; G06F 30/27

(52) U.S. Cl. CPC G16C 20/30; G01W 1/10; G06F 30/27

(57) ABSTRACT

A system for online-coupled atmospheric chemical transport modeling and data assimilation is built using online coupling and secondary parallelization. In the prototype code of a nested air quality forecasting model system, a parallel data assimilation framework routine is introduced. The system includes observation modules, model modules, and assimilation modules. The observation module is responsible for flexible access and preprocessing of various component-type observation data. The model module handles the model integration of initial fields, involving calculations of physical and chemical processes. The assimilation module performs analysis assimilation of model state variables. This system meets the requirements for coordinated assimilation of multiple chemical component variables, simultaneous introduction and flexible combination of various types of observation data, and effective handling of non-linear and non-Gaussian distribution issues in chemical assimilation.

### CROSS REFERENCE TO RELATED APPLICATIONS

This application claims priority of application No. 2024105436909 filed in China on Apr. 30, 2024 under 35 U.S.C. § 119, the entire contents of all of which are hereby incorporated by reference.

### TECHNICAL FIELD

The present invention relates to the field of aerosol chemical composition detection, particularly to a system of online coupling atmospheric chemical transport models and data assimilation.

### BACKGROUND TECHNOLOGY

Aerosols are mixtures composed of complex and diverse chemical components, where sulfate (SO42−), nitrate (NO3−), ammonium (NH4+), organic matter (OM), and elemental carbon (EC) or black carbon (BC) are key chemical components. During circulation, they enter plants, soil, water bodies, etc., impacting the chemical balance of the Earth system and ecological stability. These chemical components also have different effects on human health and climate change. Therefore, characterizing aerosol chemical composition is crucial for accurately identifying specific pollution sources, elucidating pollution causes, developing targeted emission reduction and control strategies, and assessing human health and climate effects.

Existing component detection technologies cannot describe the spatiotemporal continuous aerosol chemical composition, numerical models can characterize the spatiotemporal distribution features of various chemical components, but their uncertainties are numerous, including initial boundary conditions, physicochemical mechanisms, emission inventories, meteorological field inputs, etc., leading to significant deviations between model simulation results and observations. Data assimilation technology can effectively fuse observational data and numerical models, utilizing observation information and model information as well as their respective uncertainties to describe an atmospheric initial state as realistically as possible, improving the ability of numerical model simulation and forecasting. Research and application of vertical assimilation of aerosol chemical components at home and abroad are still blank.

### SUMMARY OF THE INVENTION

The purpose of the present invention is to solve the shortcomings existing in the prior art and provide a system for online coupled atmospheric chemical transport modeling and data assimilation. The system adopts online coupling and secondary parallel construction, including an observation module, a model module, and an assimilation module.

The observation module is responsible for flexible input and preprocessing of various component types of observation data, so that the assimilation module can utilize the observation data.

The model module is used for initializing the model integration field, calculating output forecast fields through physical and chemical processes, and providing background fields for the assimilation module.

The assimilation module analyzes and assimilates the model state variables output by the model module, improves the background field based on the observation data, generates an analysis field that is consistent with the observation data, and provides the optimal initialization field for the next model integration at the same time.

Furthermore, the system introduces a parallel data assimilation framework routine into the prototype code of a nested air quality forecasting model system, using an online coupling method. The nested air quality forecasting model system and the parallel data assimilation framework are controlled by a main program, with model integration and analysis assimilation at each time step being coherent rather than independent.

Between the nested air quality forecasting model system S and the parallel data assimilation framework, multiple ensemble members run simultaneously and exchange data through a two-level parallel method based on the message passing interface MPI, while maintaining parallelized calculations of numerical matrices within individual ensemble members.

Additionally, the system's state variables include volume concentration and RH of emissions, all of which are three-dimensional structures stored in two-dimensional matrices. The coordinate index of each grid point contains its three-dimensional information for regional localization processing.

Moreover, the system is coupled with the observation module infrastructure OMI, which serves as an extension of the parallel data assimilation framework, providing input/output interfaces for multiple sources of observation data and introducing more observation types and sources, while modularizing observation types, observation operators, and localization processing.

Preferably, the observation module infrastructure OMI provides common core routines and independent user support routines for each observation type, where the user support routines are used for reading observations, calling observation operators, and applying covariance localization.

Furthermore, the assimilation module employs a localized Kalman nonlinear ensemble transform filter LKNETF algorithm, obtaining analysis samples by using transformation matrices and the square root of prediction error covariances, indirectly updating the model state by influencing the weights of the prior ensemble with observation data, and finally performing adaptive adjustments to the system through mixed weights.

Furthermore, the localized Kalman nonlinear ensemble transform filtering LKNETF algorithm combines ensemble Kalman filtering and its variant EnKFs, ensemble transform Kalman filtering ETKF, particle filtering and its variant PFs, nonlinear ensemble transform filtering NETF. The localized Kalman nonlinear ensemble transform filtering LKNETF algorithm integrates LETKF and LNETF through a one-step process by mixing weights.

Moreover, the model module introduces a mixed nonlinear ensemble assimilation algorithm, considering nonlinear and non-Gaussian distribution disturbances for emission source inputs, perturbing the emission species based on the uncertainty of the emission species.

Preferably, a 2-D pseudo-random perturbation field is created based on the Evensen method, the original perturbation coefficient matrix is obtained through mathematical transformations of the 2-D pseudo-random perturbation field, and the perturbation coefficient matrix is calculated through the original perturbation coefficient matrix. The perturbed emission source matrix is obtained through the original emission source matrix and the perturbation coefficient matrix.

Compared to existing technology, the beneficial effects of the present invention are as follows.

(1) This invention system utilizes an online coupling method, where the model integration results of the background field can be directly passed to the parallel data assimilation framework for analysis and assimilation. This approach avoids the need for writing data exchange interfaces and executing cumbersome instructions. The parallel data assimilation framework and model form an integrated assimilation system controlled by a main program, effectively utilizing a large number of processors in high-performance computing clusters.

(2) This invention system has a two-level parallel computing framework, where internal model parallel computing and ensemble task parallel computing can be executed simultaneously, significantly improving the computational efficiency of the assimilation system.

(3) This invention system integrates a self-developed nested air quality forecast model system with a parallel data assimilation framework online, meeting the coordinated assimilation of various chemical component variables, achieving excellent chemical component assimilation effects.

### BRIEF DESCRIPTION OF THE FIGURES

The Figures are used to provide a further understanding of the present invention and form part of the specification. They are used in conjunction with the embodiments to explain the present invention and do not limit the present invention. In the Figures:

FIG. 1 shows a schematic diagram of the system structure of an online coupled atmospheric chemical transport model with data assimilation of the present invention;

FIG. 2 shows a schematic diagram of the state variable structure in the system of an online coupled atmospheric chemical transport model with data assimilation of the present invention; and

FIG. 3 presents a list of uncertainties for different emitted species of the present invention.

### DETAILED DESCRIPTION

In order to make the purpose, technical solutions and advantages of the embodiments of the present application clearer, the technical solutions in the embodiments of the present application will be described clearly and completely in the following in conjunction with the accompanying drawings in the embodiments, and it is obvious that the described embodiments are a part of the embodiments of the present application and not all of the embodiments. Based on the embodiments in this application, all other embodiments obtained by a person of ordinary skill in the art without making creative labor fall within the scope of protection of this application.

Specific embodiments of the present invention are described below in connection with the accompanying drawings as well as embodiments.

### Example of Implementation

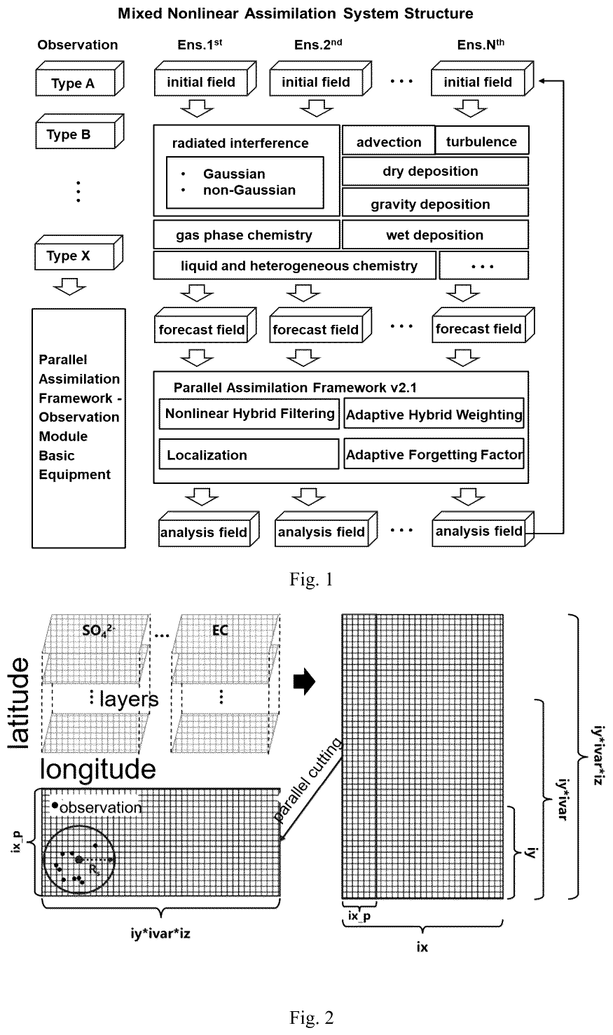

As shown in FIG. 1, a system for on-line coupling of atmospheric chemical transport modes and data assimilation is provided for the present embodiment, said system being built in an on-line coupling and secondary parallel manner, comprising: an observation module, a mode module, and an assimilation module.

The observation module is responsible for the flexible input and pre-processing of observations of multiple component types so that the observations can be utilized by said assimilation module.

The mode module is used to initialize the mode integrals of the field, calculate the output forecast field through a rationalization process, and provide a background field for assimilation module.

The assimilation module performs analytical assimilation of the mode state variables output by mode module, improves the background field based on observations, generates an analytical field coordinated with the observations, and at the same time provides an optimal initialization field for mode integration at the next moment.

Wherein this system employs online coupling by introducing parallel information assimilation framework routines in a prototype code for a nested air quality forecast model system, wherein the nested air quality forecast model system and the parallel information assimilation framework are controlled by a single master program, and wherein the model integrations and the analytical assimilations are coherent rather than independent at each time step.

Further, the nested air quality forecasting model system and the parallel information assimilation framework are based on a message passing interface MPI between the nested air quality forecasting model system and the parallel information assimilation framework, which enables multiple ensemble members to run and exchange data simultaneously by means of a second level of parallelism, while maintaining parallelized computation of numerical matrices in a single ensemble member.

Specifically, the system consists of a self-developed nested air quality forecasting model system coupled online with the parallel data assimilation framework, which is coupled in such a way that there is an integration between the nested air quality forecasting model system and the parallel data assimilation framework, implying that the model integration and analytical assimilation are continuous. Compared to offline coupling, online coupling has a wealth of advantages, such as: (1) the initialization processes of the parallel data assimilation framework and the model need to be performed only once instead of twice independently; (2) the model integration results as background fields in the online model can be directly passed to the parallel data assimilation framework for analytical assimilation, whereas the analytical assimilation results of the parallel data assimilation framework as the optimal initial field can be directly passed to the model for integration calculation, which avoids writing data exchange interfaces and executing cumbersome commands; (3) in the online mode, the parallel data assimilation framework and the model are integrated assimilation systems controlled by a master program, which can effectively utilize a large number of processors in the high-performance computing cluster.

At the same time, the system has a second-level parallel computing framework, where parallel computation within the model and parallel computation of the ensemble tasks can be executed simultaneously, thus significantly improving the computational efficiency of the assimilation system. Taking running 20 ensembles as an example, it means executing 20 modeling tasks, and each modeling task needs to perform integral computation on a large grid. With sufficient number of processors, 20 model tasks can be executed at the same time based on 2-level parallelism, and each model task can parallelize the cut computation on the large grid.

In this embodiment, the compositional structure of the described system as well as the main program architecture are demonstrated as shown in FIG. 1. The system structure can be divided into three parts, namely the observation module, the model module and the assimilation module. The observation part is responsible for the flexible access and pre-processing of multiple component types of observation data so that the assimilation system can efficiently and fully utilize the observation data. The model part is responsible for the model integration of the initial field, which involves the calculation of physical and chemical processes, such as advective transport, turbulent diffusion, dry deposition, wet deposition, gas-phase chemistry, liquid-phase chemistry, and non-homogeneous-phase chemistry, etc., and thus outputs the forecast field, which provides the background field for the analysis of assimilation process. The assimilation part is responsible for the analytical assimilation of the model state variables, improving the background field based on the observation data, generating the analytical field coordinated with the observation data, and providing the optimal initial field for the model integration at the next moment. In order to adapt to the nonlinear and non-Gaussian distribution characteristics in the chemical component assimilation, the assimilation part adopts a hybrid nonlinear assimilation scheme.

Further, the state variables in above system include NH4+, SO42−, NO3−, Na+, OC, EC, Brown Carbon BrC, Soil PM2.5, Soil PM10, Sea Salt, Mass Concentration of Fine Sand and Dust, Coarse Sand and Dust, Volumetric Concentration of SO2 and NO2, and RH, and all of the state variables are in a three-dimensional structure, and the matrices of the state variables are stored in two-dimensional form, and each of the coordinate index of a grid point contains its three-dimensional information for regional localization.

The structural design of the state variables of the present embodiment is shown in FIG. 2, and the 15 state variables are all three-dimensional in structure, with dimensional sizes of the number of grids in the longitudinal direction (ix), the number of grids in the latitudinal direction (iy), and the number of vertical layers in the vertical direction (iz), and ix, iy, and iz in the present invention are set to be 300, 249, and 40, respectively. The 15 three-dimensional state variables are converted into the parallel profile assimilation framework in the two-dimensional state variable matrix, wherein the vertical axis is arranged in the order of iy, the number of variables (ivar) and iz from inside to outside, the number of grids in the direction of the horizontal axis is ix, and the number of grids in the direction of the vertical axis is iy*ivar*iz. It is worth noting that in the described system, even though the state variable matrix is stored in the form of two dimensions, the coordinate indexes of each of the grids contain three-dimensional information in order to localize the horizontal localization and the vertical localization are treated separately. This is because the horizontal direction is isometric (i.e., the horizontal grid is at 5 km resolution) while the vertical height is non-isometric (i.e., the terrain follows the height) and there is a significant difference in the order of magnitude between the two, and therefore, the same localization radius cannot be shared.

Further, the system is coupled to the Observation Module Infrastructure (OMI), which is an extension of the parallel data assimilation framework, providing input/output interfaces to multiple sources of observations while introducing additional types and sources of observations.

The Observation Module Infrastructure OMI provides common core routines and separate user support routines for each observation type, the user support routines being used to read observations, invoke the observation operators and apply covariance localization.

Specifically, in this embodiment, OMI, as an extension of the parallel data assimilation framework, provides input/output (I/O) interfaces to multiple sources of observations to ensure that the data do not interfere with each other during reading and writing, and modularizes the observation types, observation arithmetic, and localization processing in order to simplify the user's processing of the observations. The OMI provides common core routines for each observation type and independent user support OMI provides common core routines and separate user support routines for each observation type, which are responsible for reading observations, invoking observation operators, and applying covariance localization. Note that the observation localization is further based on the regional localization by assigning different weights to the observations within the region, which is affected by the distance between the observation location and the analyzed location, and is computed by a fifth-order polynomial function. The modules of all observation types are integrated and managed by callback routines, and the simultaneous reading, writing and scheduling of different observation types are realized by defining observation indexes, which is conducive to the flexible combination and simultaneous assimilation of different observations and improves the operational efficiency, and provides a technological guarantee for the synergistic assimilation of multiple PM2.5 chemical component data.

Further, the assimilation module employs a localized Kalman nonlinear ensemble transform filtering LKNETF algorithm to obtain analytical samples by using the square root of the transform matrix and the prediction error covariance, and then indirectly updating the pattern states by using observations to influence the weights of the a priori ensemble, and finally adapting the system adaptively by mixing the weights.

In this embodiment, the localized Kalman nonlinear ensemble transformed filter LKNETF algorithm combines an ensemble transformed Kalman filter ETKF in ensemble Kalman filters and their variants EnKFs and a nonlinear ensemble transformed filter NETF in particle filters and their variants PFs, and the localized Kalman nonlinear ensemble transformed filter LKNETF algorithm combines, by mixing weights, the LETKF and the LNETF in a one-step combination.

Specifically, in this embodiment, the variational and ensemble Kalman filters and their variants EnKFs, and the particle filters and their variants PFs are the three dominant classes of assimilation algorithms, but each of them has certain limitations. Vars requires explicit computation and storage of the background field error covariance BEC matrices, which are usually difficult to compute due to the huge dimensionality of the state variables of the actual mode system. Moreover, the BEC matrix in Vars is static, which does not harmonize with dynamic mode systems. In addition, concomitant operators need to be written in the process of solving the target generalized function, which is cumbersome to implement. Unlike Vars, EnKFs do not need to compute and store the BEC matrix explicitly, but solve it in the form of a gain matrix, which does not require writing an accompanying operator, and the BEC matrix has a flow-dependent property.

ETKF is a deterministic filter in EnKFs that efficiently obtains analytical samples by utilizing the square root of the transformation matrix and the prediction error covariance. Compared with the stochastic filtering in EnKFs, ETKF avoids the underestimation of the analytical error covariance due to the observation of random perturbations. In addition, ETKF can be applied to the case of small ensemble size. The implementation of ETKF can be divided into a forecasting step and an analyzing step.

NETF is a second-order exact ensemble square-root filter that can be effectively applied to DA for nonlinear and non-Gaussian scenarios. Like traditional PF, NETF updates the model state indirectly by using the observed data to influence the weights of the prior ensemble. However, traditional PF and NETF differ in the sampling methodology. PF is based on observed data with particle weights computed using Monte Carlo and Bayesian methods, and then generates the analysis ensemble by weighting the prediction ensemble obtained from resampling. In high-dimensional systems, as DA proceeds, the difference in the weights of particles increases, and the weights of most particles tend to zero, with a few particles dominating, leading to filter degradation. In contrast, NETF generates the analysis ensemble by performing a deterministic matrix square root transformation on the forecast ensemble, and the mean and covariance matrices of the analysis ensemble are consistent with the weighting results in PF.

In this embodiment, LKNETF combines LETKF and LNETF in a “one-step” fashion by mixing the weights(γ). LETKF and LNETF are combined in a “one-step” fashion, and the localized Kalman nonlinear ensemble transform filtering LKNETF algorithm combines LETKF and LNETF in a “one-step” fashion by means of mixing weights(γ), the combination formula being as follows:

$${X}_{HSync}^{a} = \overline{{X}^{f}} + ( 1 - γ ) Δ {X}_{LNETF} + γ Δ {X}_{LETKF} ,$$

wherein the X̅^̅{̅f̅}̅ is a forecast state vector mean value, and

$${X}_{HSync}^{a}$$

is an analytical state vector of said localized Kalman nonlinear ensemble transform filtered LKNETF horizontal synchronization signal, and the mixing weight(γ) is adaptively adjusted according to the non-Gaussianity, and when the mixing weight(γ) tends to 1, the analytical increment (ΔX_{LETKF}) obtained by the LETKF that dominates, and the mode module prefers linear and Gaussian distributions. On the contrary, when the mixing weight(γ) tends to 0, the analytical increment (ΔX_{LNETF}) obtained by the LNETF that dominates, and the mode module prefers non-linear and non-Gaussian distributions.

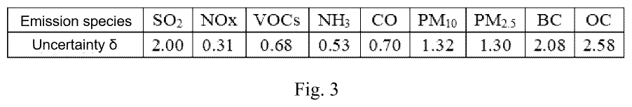

Further, a hybrid nonlinear ensemble assimilation algorithm is introduced to adapt to the nonlinear and non-Gaussian distribution case, and the inputs of the emission sources are perturbed with non-Gaussian distributions, based on the uncertainties of the emission speciesδ. As shown in FIG. 3, SO2, NOX, VOCs, NH3, CO, PM10, PM2.5, BC, and OC are perturbed, and the unperturbed original emission source matrix is Eₚ is multiplied by the matrix of perturbation coefficients corresponding to the N ensemblesθᵢ to obtain the N perturbed emission source matricesEᵢ and the equations are as follows:

$${E}_{i} = {E}_{p} \times {θ}_{i} , i = 1 , 2 , … , N ,$$

where a 2-D pseudo-randomized perturbation field Pᵢ is created based on the Evensen method. The mathematical transformation of Pᵢ is performed to obtain the original perturbation coefficient matrix θₒ_{ᵢ}. Calculate to obtain the perturbation coefficient matrixθᵢ by the original perturbation coefficient matrixθₒ_{ᵢ}, and the formula is as follows:

$$ln {θ}_{{o}_{i}} = ( \frac{( {P}_{i} - \frac{1}{N} \times {\sum }_{i = 1}^{N} {P}_{i} )}{\sqrt{\frac{1}{N} \times {\sum }_{i = 1}^{N} {( {P}_{i} - \frac{1}{N} \times {\sum }_{i = 1}^{N} {P}_{i} )}^{2}}} - \frac{1}{2} \times ln ( 1 + {δ}^{2} ) ) \times \sqrt{ln ( 1 + {δ}^{2} )} ,$$

$${θ}_{i} = \frac{( {θ}_{{o}_{i}} - \frac{1}{N} \times {\sum }_{i = 1}^{N} {θ}_{{o}_{i}} )}{\sqrt{\frac{1}{N} \times {\sum }_{i = 1}^{N} {( {θ}_{{o}_{i}} - \frac{1}{N} \times {\sum }_{i = 1}^{N} {θ}_{{o}_{i}} )}^{2}}} \times ( \frac{1}{N} \times {\sum }_{i = 1}^{N} {θ}_{{o}_{i}} ) \times δ + \frac{1}{N} \times {\sum }_{i = 1}^{N} {θ}_{{o}_{i}} ,$$

The matrix penalties in the above formulas are all Schur products, that is, the corresponding matrix elements are multiplied.

Specifically, in the ensemble assimilation of this embodiment, the ensemble members characterize the uncertainty of the model system, and the ensemble perturbations computed using the ensemble members represent the error distribution of the model state, and thus the creation of the ensemble members is crucial in the ensemble assimilation, which directly affects the assimilation effect. For atmospheric chemical transport models, emission sources serve as important input information that drives the generation and elimination evolution of model variables, which is an important source of model uncertainty. Perturbing the emission source input can fully characterize the uncertainty of aerosol emission in the model, effectively enhance the consistency of the ensemble error diffusion, and improve the aerosol assimilation effect. Previous ensemble perturbation methods create a certain number of ensemble members by perturbing the emission source inputs according to the error probability distribution function of the emission species.

Finally, it should be noted that the above is only a preferred embodiment of the present invention, and is not intended to limit the present invention. Although the present invention has been described in detail with reference to the foregoing embodiments, technical personnel in the field are still able to make modifications to the technical solutions described in the foregoing embodiments or make equivalent substitutions of some of the technical features therein. Any modification, equivalent substitution, improvement, etc. made within the spirit and principles of the present invention shall be included in the scope of protection of the present invention.

1. A system of online coupled atmospheric chemistry transport model with data assimilation including adopting online coupling and secondary parallel construction, the system comprising:
an observation module responsible for flexible input and preprocessing of various component types of observational data so that the assimilation module can utilize the observational data;
a model module configured to initiate model integration of fields, calculating output forecast fields through physical and chemical processes to provide background fields for the assimilation module, and
an assimilation module configured to:
analyze and assimilate model state variables output by the model module;
improve background fields based on observational data;
generate analysis fields coordinated with the observational data; and
provide optimal initialization fields for the model.

2. The online coupled atmospheric chemistry transport model with data assimilation system according to claim 1, further comprising introducing parallel data assimilation framework routines into a prototype code of a nested air quality forecast model system, adopting online coupled coupling, whereby the nested air quality forecast model system and the parallel data assimilation framework are controlled by a main program, and the model integration and analysis assimilation for each time step are coherent rather than independent.

3. The online coupled atmospheric chemistry transport model with data assimilation system according to claim 2, further comprising implementing multiple ensemble members to run simultaneously with data exchange through an information transfer interface MPI between the nested air quality forecast model system and a parallel data assimilation framework, maintaining parallelized calculation of numerical matrices within individual ensemble members.

4. The online coupled atmospheric chemistry transport model with data assimilation system according to claim 1, wherein state variables in the system, including the volume concentration and RH of emissions, are three-dimensional structures stored as two-dimensional matrices, with each grid point's coordinate index containing three-dimensional information for regional localization processing.

5. The online coupled atmospheric chemistry transport model with data assimilation system according to claim 1, further comprising coupling the system with an observation module infrastructure (OMI), where the OMI serves as an extension of a parallel data assimilation framework providing input/output interfaces for multiple sources of observational data:
introducing additional observation types and sources; and
modularizing observation types, observation operators, and localization processing.

6. The online coupled atmospheric chemistry transport model with data assimilation system according to claim 5, wherein the observation module infrastructure OMI provides common core routines and independent user support routines for each observation type, with user support routines used for reading observations, invoking observation operators, and applying covariance localization.

7. The online coupled atmospheric chemistry transport model with data assimilation system according to claim 1, wherein the assimilation module is configured to:
adopt a localized Kalman nonlinear ensemble transform filter LKNETF algorithm;
obtain analysis samples by utilizing the square root of transformation matrices and prediction error covariances; and
indirectly update the model state using observation data to adjust the system adaptively through mixing weights.

8. The online coupled atmospheric chemistry transport model with data assimilation system according to claim 7, further comprising, through a localized Kalman nonlinear ensemble transform filter LKNETF algorithm, combining an ensemble transform Kalman filter ETKF and a nonlinear ensemble transform filter NETF from ensemble Kalman filters and their variants as well as particle filters and their variants, achieving a one-step combination of LETKF and LNETF through mixing weights.

9. The system of online-coupled atmospheric chemistry transport model with data assimilation, as claimed in claim 1, further comprising:
introducing a mixed nonlinear ensemble assimilation algorithm;
considering nonlinear and non-Gaussian distribution perturbations for emission source inputs; and
perturbing emission species based on their uncertainties.

10. The system of online-coupled atmospheric chemistry transport model with data assimilation as claimed in claim 9, further comprising
creating a 2-D pseudo-random perturbation field based on the Evensen method;
mathematically transforming the 2-D pseudo-random perturbation field to obtain an original perturbation coefficient matrix;
calculating a perturbation coefficient matrix from the original perturbation coefficient matrix; and
obtaining a perturbed emission source matrix using the original emission source matrix and the perturbation coefficient matrix.
